(19) United States
(12) Patent Application Publication
(10) Pub. No.: US 2024/0343763 A1
(43) Pub. Date: Oct. 17, 2024

(54) DETOXIFIED GLUTEN PROTEIN FOR THE FORMULATION OF FOODS FOR SPECIAL MEDICAL PURPOSES

(71) Applicant: ENEA - AGENZIA NAZIONALE PER LE NUOVE TECNOLOGIE, L'ENERGIA E LO SVILUPPO ECONOMICO SOSTENIBILE, Rome (IT)

(72) Inventors: Selene BASCHIERI, Trevignano Romano (RM) (IT); Marcello DONINI, Roma (RM) (IT); Chiara LICO, Roma (RM) (IT); Carla MARUSIC, Roma (RM) (IT); Silvia MASSA, Roma (RM) (IT)

(21) Appl. No.: 18/294,018

(22) PCT Filed: Jul. 21, 2022
(86) PCT No.: PCT/IB2022/056735

§ 371 Date: Jan. 31, 2024

(30) Foreign Application Priority Data
Aug. 4, 2021 (IT) 102021000021194

(51) Int. Cl. C07K 14/415; A21D 13/066; A23L 5/20; C12N 15/82

(52) U.S. Cl. CPC C07K 14/415; A21D 13/066; A23L 5/20; C12N 15/8242

(57) ABSTRACT

A modified α-gliadin (polypeptide) free of toxicity or with less toxicity as compared to the predominant wild-type form, ideal for the preparation of food intended for individuals suffering from celiac disease or individuals prone to developing the disease. The invention further relates to DNA sequences encoding the modified α-gliadins, organisms capable of producing such modified α-gliadins and products including such modified α-gliadins.

### FIELD OF APPLICATION

The present invention relates to a modified α-gliadin (polypeptide) free of toxicity or with reduced toxicity with respect to the predominant wild-type (WT) form, ideal for the preparation of food intended for individuals suffering from celiac disease or individuals prone to developing the disease. The invention further relates to DNA sequences encoding the modified α-gliadins, organisms capable of producing such modified α-gliadins and products comprising such modified α-gliadins.

### Background Art

Celiac disease is an immune-mediated disease which affects about 1% of the population in Europe. The disease is characterized by a state of chronic inflammation of the small intestine triggered by the ingestion of gluten proteins (gliadins and glutenins) present in the caryopsis or kernel, i.e., the dried fruit, of some grains, including wheat, in genetically predisposed subjects, because they carry specific allelic combinations of the human leukocyte antigen system (HLA) (DQ2.5, DQ8, DQ2.2 or DQ7.5).

The autoantigen to which, at the intestinal level, the auto-antibody response is activated is tissue transglutaminase, a calcium-dependent membrane enzyme. The auto-antibodies cause the inactivation of such an enzyme, inhibit the differentiation of enterocytes and flatten the epithelium of intestinal villi, causing the malabsorption of nutrients.

The diagnosis of the disease involves serological investigations, intestinal biopsy and genetic screening. Serum assays predict the dosage of class A antibodies (IgA) against tissue transglutaminase (anti-tTG), anti-endomysium (EMA) and anti-gliadin (AGA). The class G AGAs (IgG) are a less specific marker. In early childhood, AGAs have a higher sensitivity than anti-tTGs, but the AGA test loses sensitivity with advancing age.

There are various types of celiac disease: typical or symptomatic, atypical, silent and potential. If left untreated, the disease increases the likelihood of contracting other diseases, and the complete and permanent elimination of gluten from the diet is the only treatment currently available to achieve symptom remission and the prevention of complications. The gluten elimination must be very strict and must be carried out scrupulously throughout life, since even minimal amounts of gluten are sufficient to regress the patient's improved health conditions with the diet or to prevent their recovery. In addition to wheat and spelt, similar grain species, such as barley, rye, triticale and Khorasan wheat, also contain gluten proteins. Other grains, such as corn, millet, sorghum, teff, rice and zizania, are considered safe, as are the pseudo-grains amaranth, quinoa and buckwheat.

The physiological function of gliadins and glutenins (a mixture of more than 100 different proteins) is that of energy reserve and support of germination and early stages of plant development. When water is added to the flour obtained by grinding the kernels to make dough, the matrix which such proteins form around the starch granules transforms into an elastic and viscous network called gluten. In pasta, during cooking, the gluten network slows the absorption of water by the starch, giving the product tenacity and elasticity, while in bread it retains the gas bubbles produced by the yeast; this property, combined with cohesion, homogeneity, viscoelasticity and tenacity, allows obtaining a soft and elastic product, pleasant to the palate.

The quality of life of celiac patients is lower than that of healthy subjects because following a well-balanced and strict gluten-free diet is very difficult. In fact, the grains celiacs cannot eat are found in many products and the risk of accidental contamination along the food chain is very high. Furthermore, gluten is often added in the industrial production of many foods to improve the features thereof in terms of consistency or density. The products currently on the market can be divided into three main categories: i) allowed foods because they are naturally gluten-free (gluten content less than 20 parts per million, p.p.m.; maximum limit recognized as a tolerable amount of gluten for a celiac); ii) at-risk foods because they have a gluten content between 20 p.p.m. and 100 p.p.m. or at risk of contamination and for which it is necessary to know and control the ingredients and processing processes; iii) prohibited foods because they contain gluten.

There are numerous, quite diversified gluten proteins, and they are encoded by several genes grouped in loci distributed on different chromosomes. Such a genetic complexity makes it impossible to generate gluten-free wheat or other grains using classic genetics techniques, such as cross-breeding and selection.

Although considerable progress has been made in improving the palatability of gluten-free foods, the industrial products available on the market are often highly caloric, have a lower nutritional value and are particularly expensive. Gluten-free products with good nutritional properties can be obtained using the seeds of lupin, hemp, soybeans, corn, peas, but the doughs obtained with the flours prepared from these seeds or kernels do not have the features required by the bakery and pasta industry. The replacement of the gluten network is currently one of the biggest challenges in food technology. To eliminate gluten toxicity, it is mainly necessary to eliminate the toxicity of α-gliadins.

Patent application WO 2011/157806 describes a process for making modified α-gliadins in which one or more substitutions, or deletions relate to at least one of the epitopes DQ2 (DQ2-Glia-α1, DQ2-Glia-α2 and/or DQ2-Glia-α3) in the immunodominant region of the protein (peptide 33, amino acid positions 56-88) and/or the epitope DQ8 Glia-α1 (amino acid positions 229-237). The substitutions, or deletions, relate to the amino acid in position 3 and/or 8 of the epitope DQ2-Glia-α1: Pro-{Phe/Tyr}-Pro-Gln-Pro-{Gln/Glu}-Leu-Pro-Tyr, the amino acid in position 3 and/or 8 of the epitope DQ2-Glia-α2: {Pro/Phe}-Pro-Gln-Pro-{Gln/Glu}-Leu-Pro-Tyr-Pro-Gln, the amino acid in position 3 and/or 8 of the epitope DQ2-Glia-α 3 Phe-Arg-Pro-Gln-Gln-Pro-Tyr-Pro-Gln and the amino acid in position 3 and/or 5 of the epitope DQ8 Glia-α1: Gln-Gly-Ser-Phe-Gln-Pro-Ser-Gln-Gln. In the case of the three DQ2 epitopes, preferably the amino acid in position 3 and/or 8 is replaced with a serine while in the case of the DQ8 epitope the amino acid in position 3 is preferably replaced with phenylalanine and the one in position 5 with arginine. The elimination of immunogenicity which would be achieved by these modifications was not verified using the recombinant modified protein but by in vitro assays using synthetic peptides.

Patent Application WO 2021/001784 describes a process for making modified forms of α-gliadin which bind to T cells derived from a celiac patient with less affinity as compared to the corresponding non-mutated α-gliadin. Also in this case the mutations relate to localized amino acid positions between the amino acid Leu and the amino acid Phe of the peptide 33 of α-gliadin where antigenic units of 7 residues were identified (Gln-Leu-Pro-Tyr-Pro-Gln-Pro; Gln-Leu-Pro-Tyr-Ser-Gln-Pro; Pro-Leu-Pro-Tyr-Pro-Gln-Pro). In particular, the substitutions affect two or three amino acids in positions 1, 4 and 5 of the antigenic units. The substitution in position 1 comprises a substitution with a positively charged amino acid, e.g., histidine or lysine. The substitution in position 4 of the antigenic unit comprises a substitution with a proline, an aliphatic amino acid, a polar amino acid or glycine and the substitution in position 5 of the antigenic unit comprises a substitution with a small amino acid, a polar amino acid or an aromatic amino acid. In some embodiments, position 3 of the modified α-gliadin antigenic unit is also replaced with an aromatic or polar amino acid. The reduced ability of modified forms of gliadin to bind HLA was determined in accordance with a predictive computational model and not on actual protein samples. The failure to activate the T cells in biopsy samples from celiac patients was determined by detecting the levels of IFN-γ produced in an ELISA assay in response to stimulation with synthetic peptides and not with the complete modified protein.

Therefore, although it has been shown that targeted amino acid substitutions in synthetic peptides with a sequence corresponding to that of the epitopes included in P33 inhibit the in vitro stimulation of the T lymphocytes of celiac subjects, the same has never been verified with a complete α-gliadin containing such substitutions, or other modified residues outside the immunodominant region.

Therefore, despite the progress made in recent years also by virtue of recombinant DNA technology, having forms of α-gliadin free of toxicity or with reduced toxicity for formulating food for celiac patients which allow a more varied and quality diet still remains an open challenge.

### SUMMARY OF THE INVENTION

The present invention responds to the background and other requirements by providing a form of the α-gliadin protein of Triticum aestivum belonging to the locus Gli-A2 (UniProtKB-P02863-GliaWT) modified in sequence (GliaMUT) and characterized by reduced immunogenicity and good expression ability, in particular in bacteria.

The modified form of the α-gliadin protein (GliaMUT, SEQ ID No: 1) differs from the sequence of the predominant form of α-gliadin (GliaWT, SEQ ID No: 2) for 36 of the 266 amino acids.

The invention further relates to the polynucleotide sequence encoding the modified α-gliadin (gliamut, SEQ ID No: 3) according to the invention and the use thereof for producing the recombinant modified protein and/or for expressing the modified protein in plants.

Furthermore, the invention also relates to the use of the polynucleotide sequence encoding the modified α-gliadin (gliamut, SEQ ID No: 3) for the production of recombinant, non-toxic, or less harmful gluten proteins for celiac patients and/or to transform plants to produce non-toxic, or less harmful, gluten for celiac subjects.

According to a further aspect the invention also relates to food products comprising the modified α-gliadin of the invention.

### BRIEF DESCRIPTION OF THE DRAWINGS

FIG. 1 shows the alignment of the amino acid sequence of the GliaWT protein (SEQ ID No.2) (top row) and the amino acid sequence of the GliaMUT protein (SEQ ID No.19 (bottom row).

FIG. 2 shows the alignment of the nucleotide sequence of the Gliamut construct (SEQ ID No.3) (top row) and the amino acid sequence of the GliaMUT construct (SEQ ID No.14) (bottom row) with single letter code). The sequence highlighted in yellow indicates the signal peptide, the sequence highlighted in grey indicates the Factor Xa cutting site, the sequence highlighted in magenta indicates the FLAG tag sequence, the sequence highlighted in turquoise indicates the 6XHis tag sequence.

FIG. 3: shows the alignment of the nucleotide sequence of the Gliawt construct (SEQ ID No.4) (top row) and the amino acid sequence of the GliaWT construct (SEQ ID No.15) (bottom row, with single letter code) according to the invention. The sequence highlighted in yellow indicates the signal peptide, the sequence highlighted in grey indicates the Factor Xa cutting site, the sequence highlighted in magenta indicates the FLAG tag sequence, the sequence highlighted in turquoise indicates the 6XHis tag sequence.

FIG. 4: Comparison of the expression of the GliaWT and GliaMUT proteins in different strains of Escherichia coli induced for different times and at different temperatures.

FIG. 5: Comparison of the yield of the GliaWT and GliaMUT protein extraction process from the pellets of induced and non-induced bacterial cultures (N.I., used as a control).

FIG. 6: Purification of the proteins from alcoholic extracts by immobilized metal ion affinity chromatography (IMAC) under denaturing conditions. Western blot analysis of the GliaWT (6A) and GliaMUT (6B) fractions eluted by column. Purified fractions of GliaMUT revealed by Coomassie blue staining (6C).

FIG. 7: Analysis by ELISA enzyme immunoassay of the recognition of the GliaMUT and GliaWT proteins in bacterial extracts (7A) or after purification (7B) by the monoclonal antibody R5 specific for the peptide P33.

FIG. 8: Analysis by ELISA enzyme immunoassay of the recognition of the GliaMUT and GliaWT proteins by a polyclonal antibody of anti-gliadin rabbit (8A) and of antibodies present in the certified serum of a celiac subject at different dilutions (8B).

FIG. 9: Western blot analysis of the expression of GliaWT (9A) and GliaMUT (9B) in Nicotiana benthamiana and the fractions (E3, E4, E5, E6) of GliaMUT purified by IMAC (9C).

### DETAILED DESCRIPTION OF THE INVENTION

It is the object of the present invention to provide a modified form (GliaMUT) of the α-gliadin protein sequence of Triticum aestivum (UniProtKB-P02863-GliaWT) characterized by lack of toxicity, or reduced toxicity, for the celiac patient.

The modified form of the α-gliadin protein (GliaMUT) according to the invention has sequence identity between 80% and 90% with the sequence of the predominant form of α-gliadin (GliaWT), preferably the two sequences share a degree of homology between 84-87%, even more preferably a degree of homology equal to 86.5% (230/266). The modified form of the α-gliadin protein (GliaMUT) according to the invention differs from the sequence of the predominant form of α-gliadin (GliaWT) for 36 of the 266 amino acids and has sequence SEQ ID No: 1.

The terms “polypeptide,” “peptide,” and “protein” are used interchangeably herein to refer to an amino acid polymer. The terms apply to amino acid polymers in which one or more amino acid residues is an artificial chemical mimetic of a corresponding natural amino acid, as well as to natural amino acid polymers and non-naturally occurring amino acid polymers. The term “amino acid” refers to natural and synthetic amino acids, as well as amino acid analogues and amino acid mimetics which function similarly to natural amino acids. Natural amino acids are those encoded by the genetic code, as well as amino acids which are then modified. Amino acid analogs refer to compounds having the same basic chemical structure as a natural amino acid, i.e., a carbon bonded to a hydrogen, a carboxy group, an amino group and an R group, e.g., homoserine, norieucine, methionine sulfoxide, methionine methyl sulfonium. Such analogs have modified R groups (e.g., norieucine) or modified peptide backbones, but retain the same basic chemical structure as a natural amino acid. Amino acid mimetics refer to chemical compounds which have a different structure from the general chemical structure of an amino acid, but which function similarly to a natural amino acid. In the present description, amino acids are indicated by their three-letter symbols commonly known to those skilled in the art or by one-letter symbols recommended by the IUPAC-IUB Biochemical Nomenclature Commission. The term “conservatively modified variants” as used herein applies to both amino acid and nucleic acid sequences.

As for amino acid sequences, those skilled in the art will recognize that individual substitutions, deletions, or additions to a nucleic acid, peptide, polypeptide, or protein sequence which alter, add, or delete a single amino acid or a small percentage of amino acids in the encoded sequence, is a “conservatively modified variant” if the alteration results in the substitution of an amino acid with a chemically similar amino acid. Conservative substitution tables which provide functionally similar amino acids are well known in the art. Conservatively modified variants do not exclude polymorphic variants, interspecies homologs, and alleles of the invention. The following eight groups each contain amino acids which are conservative substitutions for each other: 1) Alanine (A), Glycine (G); 2) Aspartic acid (D), Glutamic acid (E); 3) Asparagine (N), Glutamine (Q); 4) Arginine I, Lysine (K); 5) isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)).

Surprisingly, it has been found that substitutions of 36 amino acid residues, which result in lack of toxicity, or reduced toxicity, for the celiac patient according to the present invention, are not located exclusively in the immunodominant region of the protein (P33), but are distributed throughout the amino acid sequence of the protein.

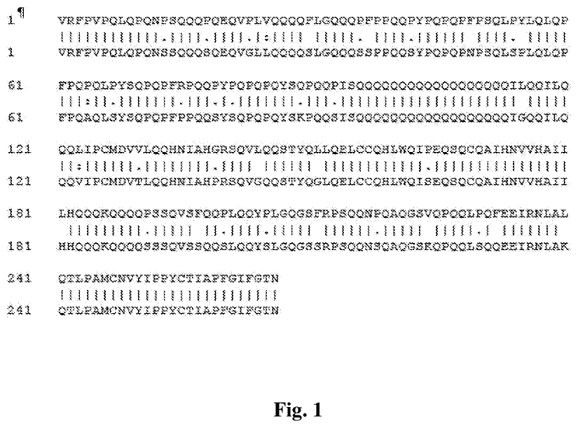

In a preferred embodiment of the invention the substitutions relate to amino acids at positions 13, 18, 23, 25, 30, 36, 37, 42, 49, 54, 55, 64, 67, 75, 79, 81, 89, 93, 115, 123, 130, 139, 144, 151, 164, 181, 191, 197, 200, 205, 211, 218, 224, 230, 232, 240 of the sequence of the predominant mature form (without signal peptide) of α-gliadin (GliaWT). 50 to 70% of the substitutions relate to conservative amino acid substitutions with respect to the sequence of the predominant form of α-gliadin (GliaWT), i.e., an amino acid substituted with another having a side group R with similar biochemical properties; preferably 64% (23/36) of the amino acid substitutions are conservative substitutions, as shown in the alignment in FIG. 1 (vertical dash: identity; two points or point: conservative substitution; no symbol: divergence).

Within the scope of the invention also are amino acid sequences which share at least 80% homology with the GliaMUT sequence and, in particular, amino acid sequences in which up to 20% of the substituted amino acids with respect to the sequence of the mature GliaMUT protein (without signal peptide) relate to amino acids at positions 13, 18, 23, 25, 30, 36, 37, 42, 49, 54, 55, 64, 67, 75, 79, 81, 89, 93, 115, 123, 130, 139, 144, 151, 164, 181, 191, 197, 200, 205, 211, 218, 224, 230, 232, 240 or modified forms of the α-gliadin protein comprising amino acid substitutions at least 80% of positions 13, 18, 23, 25, 30, 36, 37, 42, 49, 54, 55, 64, 67, 75, 79, 81, 89, 93, 115, 123, 130, 139, 144, 151, 164, 181, 191, 197, 200, 205, 211, 218, 224, 230, 232, 240 of sequence SEQ ID NO. 1.

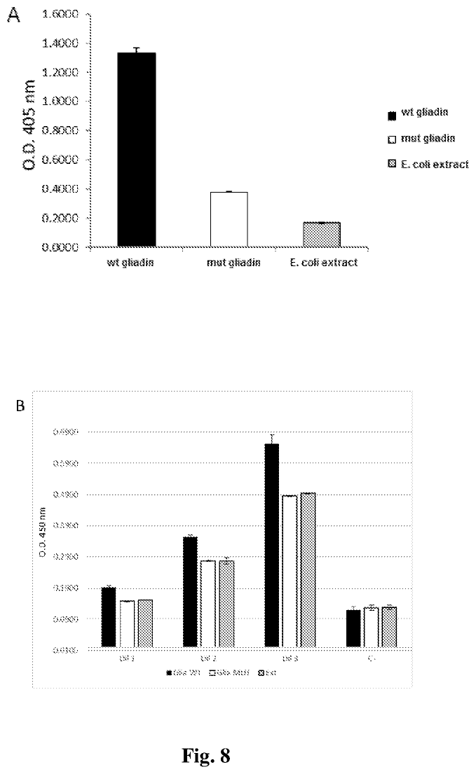

The amino acid substitutions of the GliaMUT protein have an effect on the ability to interact with a commercial rabbit anti-gliadin polyclonal antibody (G9144Anti-Gliadin (Wheat) antibody-Sigma), and with AGA antibodies present in the serum of a subject diagnosed with celiac disease but not yet on a gluten-free diet, as described later in the experimental section. In fact, the results show that the mutated α-gliadin protein according to the invention (GliaMUT), unlike the predominant WT form (GliaWT), is not recognized by the rabbit polyclonal antibody nor by the antibodies present in the serum of patients. (FIG. 8). The epitopes recognized by AGA have been shown to overlap or be closely associated with those recognized by T lymphocytes (Bateman et al, Gut 53:1274-1278, 2004; Fleur du Pré et al, Best Practice and Research Clinical Gastroenterology 29:413-423, 2015). Therefore, the fact that the GliaMUT is no longer recognized by AGA can be considered an indication that the targeted amino acid substitutions made are actually able to reduce the immunogenicity thereof.

The term “immunogenic” means the ability of an agent to give rise to an immune response in a host, both humoral and cell-mediated. Immunogenic agents are typically “foreign” to the host, for example belonging to a different species, or to a bacterium, virus or fungus. A non-extraneous agent can be immunogenic, for example in the case of an autoimmune response. “Antigen” is defined as a molecule or a portion of molecule, polypeptide, glycoprotein, lipoprotein, lipid, carbohydrate or other agent which is recognized as “foreign” and bound by an antibody and/or T cell receptor. The term “derived from” means the antigen essentially as it is in the natural antigenic context thereof, or that which has been modified to be expressed under certain conditions, to include only the most immunogenic portion or to remove other potentially harmful associated components.

The epitope (or antigenic determinant) is that small part of the antigen which binds the specific antibody or T lymphocyte antigen receptor. A single antigen molecule can contain different epitopes recognized by different antibodies or T lymphocyte antigen receptors.

An object of the invention relates to the polynucleotide sequence of DNA, deoxyribonucleic acid, having sequence SEQ ID No: 3 (gliamut) encoding the modified α-gliadin (GliaMUT) according to the invention.

The invention further comprises polynucleotide sequences encoding a modified form of α-gliadin of Triticum aestivum related due to genetic code degeneration or comprising codons optimized for expression in cell systems or host cells for industrial production.

The polynucleotide sequence was designed to recombinantly produce the GliaMUT protein.

The term “recombinant” or “chimeric” as used herein with reference, for example, to a nucleic acid, protein or vector, means that the nucleic acid, protein or vector, has been modified by the introduction of a heterologous nucleic acid or protein, or by the alteration of a native nucleic acid or protein. Thus, for example, the chimeric and recombinant vectors comprise nucleic acid sequences which are not within the native (non-chimeric or non-recombinant) form of the vector.

The terms “nucleic acid” and “polynucleotide” are used interchangeably herein to refer to polymers of deoxyribonucleotides or ribonucleotides in single- or double-stranded form. The terms comprise genes, cDNA, RNA, and oligonucleotides. Also included are nucleic acids containing known nucleotide analogues or modified residues or structural bonds, which are synthetic, naturally occurring and non-naturally occurring, which have binding properties similar to those of the reference nucleic acid and which are metabolized in a manner similar to the reference nucleotides. Unless otherwise indicated, a particular nucleic acid sequence further comprises conservatively modified variants thereof (e.g., degenerated codon substitutions) and complementary sequences, in addition to the explicitly indicated sequence. In particular, degenerate codon substitutions can be obtained by generating sequences in which the third position of one or more (or all) selected codons is substituted while encoding the same amino acid. As is known to those skilled in the art, due to the degeneration of the genetic code, a large number of functionally identical nucleic acids encode a certain protein and are to be considered within the claimed scope of protection (Batzer et al, Nucleic Acid Res. 1991, 19:5081; Ohtsuka et al., J. Biol. Chem. 1985, 260:2605-2608; Rossolini et al., Mol. Cell. Probes 1994, 8:91-98).

The polynucleotide sequence was designed using the codons preferentially used by bacterial cells, in particular Escherichia coli, to encode the amino acids.

At the 3′ end of the polynucleotide sequence, i.e., the one encoding the C-terminal end of the GliaMUT protein, there is the sequence encoding the molecular FLAG and 6XHis markers (tags), which allow detecting the recombinant proteins trough the use of antibodies and purified by affinity chromatography.

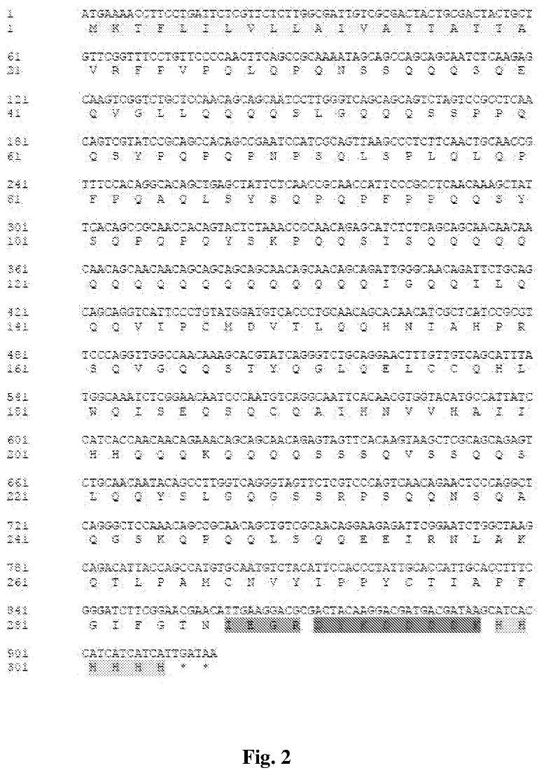

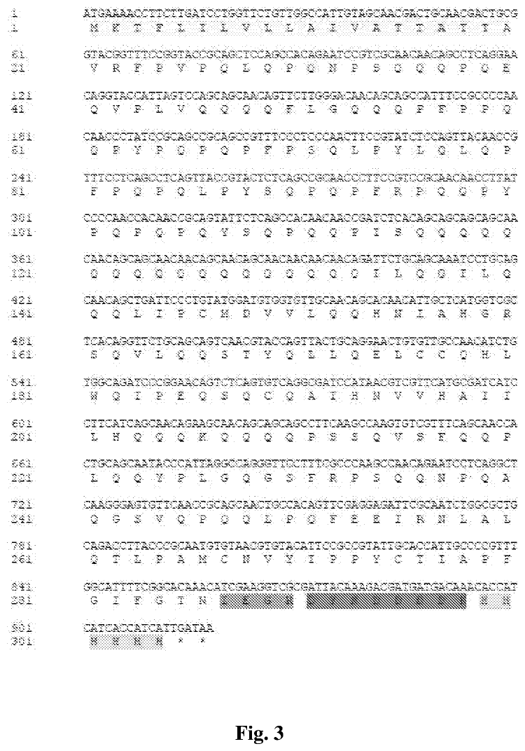

Furthermore, in the polynucleotide sequence SEQ ID No: 3, the sequence encoding the amino acid sequence recognized by the proteolytic enzyme Factor Xa is inserted between the sequence encoding the protein of interest and those encoding the tags, useful for removing the tags from the recombinant proteins. FIG. 2 shows the alignment of the nucleotide sequence (upper row) encoding the amino acid sequence (lower row, single letter code) of the GliaMUT protein according to the invention and FIG. 3 the alignment of the nucleotide sequence (upper row) encoding the amino acid sequence (lower row, single letter code) of the GliaWT protein. In FIGS. 2 and 3 the sequence highlighted in yellow indicates the secretion signal sequence, the sequence highlighted in grey indicates the Factor Xa cutting site, the sequence highlighted in magenta indicates the FLAG tag sequence, the sequence highlighted in turquoise indicates the 6XHis tag sequence.

The polynucleotide sequence SEQ ID No: 3 encoding the modified α-gliadin (GliaMUT) according to the invention bears at the 5 ′and 3′ ends consensus sequences recognized by restriction enzymes useful for insertion in plasmid expression vectors, such as pET21b (NOVAGEN) for expression in E. coli, and pBI-Ω for transient expression in the plant mediated by Agrobacterium tumefaciens, as described in Marusic C. et al. (BMC biotechnology 2007, 7:12).

According to the same design, a similar polynucleotide sequence encoding the α-gliadin protein of Triticum aestivum (UniProtKB-P02863) (GliaWT) was also produced.

The polynucleotide sequence according to the invention can be produced by synthesis or using standard molecular biology techniques known to those skilled in the art. The polynucleotide sequence according to the invention can be inserted in the expression vector, using standard molecular biology techniques and obtaining a recombinant vector used to transform competent bacterial cells, e.g., the commercial strains Shuffle (NEB) and Rosetta (NOVAGEN) and A. tumefaciens, e.g., A. tumefaciens strain LBA4404, and produce the protein.

The term “expression vector” means a nucleic acid construct, generated “recombinantly” or synthetically, with a number of specified nucleic acid elements which allow the transcription of a particular nucleic acid in a host cell. The expression vector can be part of a plasmid, virus, or nucleic acid fragment. Typically, a nucleic acid bound to a promoter to be operatively transcribed is inserted or cloned in the expression vector.

The invention also relates to the vector comprising the polynucleotide sequence according to the invention of sequence SEQ ID No: 3, which is a suitable vector for sequence expression in prokaryotic microorganisms (e.g., E. coli and Bacillus subtilis), in eukaryotic expression systems, e.g., yeasts such as Saccharomyces cerevisiae and Pichia pastoris, in eukaryotic, animal and plant expression systems. It will be apparent to those skilled in the art that the recombinant protein according to the invention can be expressed in both microorganisms and in eukaryotic organisms, for example in plant organisms.

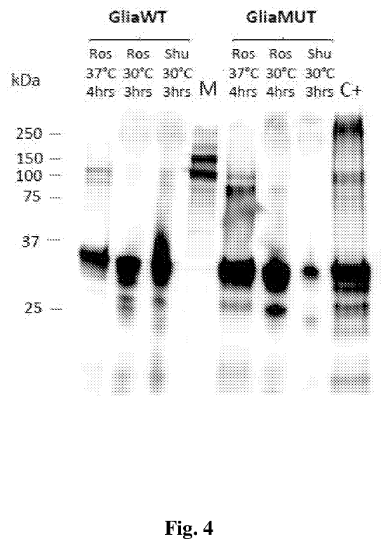

The trial carried out demonstrated that the recombinant vector comprising the polynucleotide sequence according to the invention having sequence SEQ ID No: 3 allows producing the GliaMUT protein according to the invention having sequence SEQ ID No: 1 in different strains of E. coli after induction with 0.5 mM isopropylthiogalactoside (IPTG). The experimental data showed that the mutated form of gliadin is expressed in greater amounts in the Rosetta strain of E. coli at the incubation temperature of 37° C. (FIG. 4).

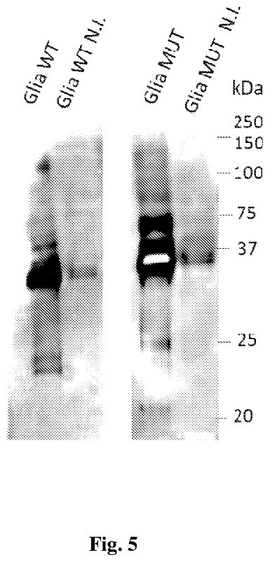

Furthermore, the recombinant GliaMUT protein according to the invention, of the expected molecular weight of 35 kDa, can be easily extracted and purified from the bacteria used for production by a precipitation method based on the use of an alcoholic solution according to the protocol developed by Molberg O et al. (Gastroenterology 125:337-344, 2003), as illustrated in the experimental section of this description. The results are shown in FIG. 5.

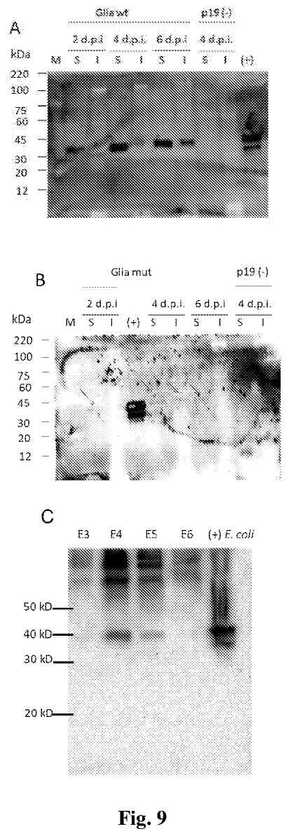

A recombinant vector for expression in plant cells comprising the polynucleotide sequence according to the invention having sequence SEQ ID No: 3 has been shown to allow producing the protein even in a system for heterologous plant expression, in fact it can also be used for the transient expression of the protein in Nicotiana benthamiana, a tobacco-like species, highly efficient in terms of yield and production times in a process on a preparatory scale. The transient expression in Nicotiana benthamiana is a reliable and linearly scalable process simply by increasing the number of plants used. The analysis showed that the GliaMUT protein accumulates in both the soluble and insoluble fraction of Nicotiana benthamiana leaf extract with a peak in both cases around 6 days post-infiltration (d.p.i.) (FIG. 9).

According to a further aspect, the invention also relates to a cell or a plant comprising in the genome thereof the sequence encoding the modified α-gliadin according to the invention. In an embodiment the cell is a cell of a grain which does not endogenously contain gluten, in particular a cell of rice (Oryza sativa), oats (Avena sativa), corn (Zea mays), sorghum (Sorghum vulgare), buckwheat (Fagopyrum esculentum), quinoa (Chenopodium quinoa) or even a cell of a genetically modified wheat species in which the genes of the α-gliadins have been eliminated. Preferably the cell comprising the sequence encoding the α-gliadin according to the invention is a rice cell.

The invention relates to the use of the modified α-gliadin protein extracted and/or purified from bacteria, eukaryotic cells and plant cells alone or in addition to flours obtained from endogenously gluten-free plant species, in particular rice (Oryza sativa), oats (Avena sativa), corn (Zea mays), sorghum (Sorghum vulgare), buckwheat (Fagopyrum esculentum), Quinoa (Chenopodium quinoa) or also from genetically modified wheat species in which the genes of α-gliadins have been eliminated to produce detoxified or reduced-toxicity foods or food products for celiac subjects.

The processed food or food product comprising the modified α-gliadin according to the invention is a product obtained by baking, a bakery product, even more preferably the processed product is bread and/or pasta.

The processed product of the grain, such as flour, is produced by grinding the caryopsis of the grain plant, which comprises in the genome thereof the polynucleotide sequence encoding the modified α-gliadin according to the invention.

The processed product, such as flour, is obtained from the seeds of a transgenic plant expressing the polynucleotide sequence encoding the modified α-gliadin, e.g., Oryza sativa.

Preferably the processed product is a product obtained by baking, a bakery product, even more preferably the processed product is bread and/or pasta.

The invention will now be further described through examples which will allow those skilled in the art to understand the advantages deriving from the use of the invention.

### EXAMPLES

### Example 1—Expression Assay in E. coli

The polynucleotide sequences SEQ ID No.3 and SEQ ID No.4 respectively encoding the GliaMUT Seq ID No. 1 and GliaWT Seq ID No. 2 proteins, obtained by synthesis (Eurofins Genomics, Germany) were cloned in the commercial expression plasmid vector for expression in E. coli pET21b (NOVAGEN).

The expression was induced by adding 0.5 mM IPTG to the culture medium at 30° C. (Shuffle and Rosetta strains) and at 37° C. (Rosetta strain), verifying the accumulation of recombinant proteins 1, 2, 3 and 4 hours after induction.

Protein extracts (5 μl) of the recombinant gliadins expressed in the Shuffle and Rosetta strains of E. coli at different temperatures were separated by SDS-PAGE 12% w/v. polyacrylamide gel electrophoresis. The proteins were electrotransferred from the gel onto PVDF membrane. The membrane was saturated using a 4% (w/v) solution of skim milk in phosphate buffer (PBS). The presence of the recombinant proteins was detected by incubating the membrane first with an anti-His tag mouse antibody at a concentration of 10 μg/ml in a 2% (w/v) solution of skim milk in PBS and then with an anti-HRP goat antibody at a dilution of 1:5000 always in a 2% (w/v) solution of skim milk in PBS. The detection was carried out by chemiluminescence with the ECL solution. A previously quantified extract of GliaWT (200 ng) was used as a positive control (C+).

Western Blot (WB) analysis demonstrated that the proteins are expressed in both strains and that the mutant protein is better expressed in the Rosetta strain (FIG. 4).

### Example 2—Alcoholic Extraction of the Recombinant Proteins Expressed in E. coli Bacteria

The pellets obtained after 4 hours of culture induction (250 ml) of the bacteria carrying the sequences coding GliaWT and GliaMUT were used to extract the proteins following the protocol of Molberg et al. The pellet was resuspended in 10 ml extraction buffer (70% EtOH, 1% DTT) left in incubation for 2 hours at 60° C. After boiling for 5 min and centrifugation (30 min at 14500×g, 12° C.), two volumes of precipitation buffer (1.5M NaCl) were added to the supernatant. After incubation o.n. at 4° C., the sample was centrifuged (30 min. at 14500× g. 12° C.) and the pellet resuspended in 2 ml of 6 M Urea, 10 mM Tris-HCl pH 8.0. 10 ml aliquots of the protein samples thus obtained were separated by SDS-PAGE 12% w/v polyacrylamide gel electrophoresis. As a negative control, the same extraction procedure was applied to pellets of non-induced bacterial cultures. The proteins were electrotransferred from the gel onto PVDF membrane. The membrane was saturated using a 4% (w/v) solution of skim milk in phosphate buffer (PBS). The presence of the recombinant proteins was detected by incubating the membrane first with an anti-Histag mouse antibody at a concentration of 10 mg/ml in a 2% (w/v) solution of skim milk in PBS and then with an anti-HRP goat antibody at a dilution of 1:5000 always in a 2% (w/v) solution of skim milk in PBS. The detection was carried out by chemiluminescence with the ECL solution (FIG. 5).

### Example 3—Purification of Extracted Recombinant Proteins

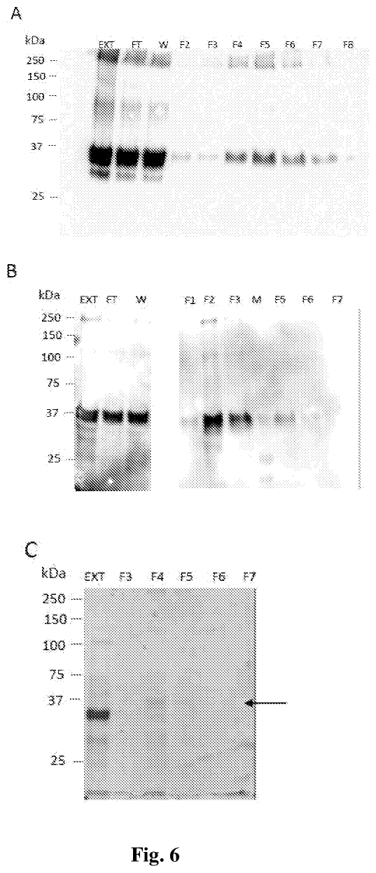

The extracts obtained from bacterial pellets (500 ml of induced culture) were used to perform purification tests of GliaWT and GliaMUT by immobilized metal ion affinity chromatography (IMAC) under denaturing conditions, i.e., using urea-containing buffers. The chromatographic column (BIORAD Bio-Scale Mini Profinity IMAC Cartridges of 1 mL volume) was equilibrated at room temperature in denaturing wash buffer 1 (6 M Urea, 300 mM KCl, 50 mM KH₂PO₄, 5 mM imidazole) at a flow of 1 ml/min. The protein-containing sample, diluted 1:10 in denaturing wash buffer 1 (5 ml final), was loaded onto the column at a flow of 1 ml/min. A first wash (10 ml) was then performed using denaturing wash buffer 1 and a second wash with equal volume of denaturing wash buffer 2 (6 M Urea, 300 mM KCl, 50 mM KH₂PO₄, 10 mM imidazole). The elution was performed with 5 ml of elution buffer (6 M Urea, 300 mM KCl, 50 mM KH₂PO₄, 250 mM imidazole) collecting 0.5 ml volume fractions. The ten fractions collected were then analyzed by WB to verify the presence of the recombinant protein (GliaWT in FIG. 6A and GliaMUT6B, respectively). The analysis of the purified fractions of GliaMUT after electrophoresis on colored polyacrylamide gel by Coomassie showed the presence of a band of the expected molecular weight of about 35 kDa (FIG. 6C).

### Example 4—Determination of the Interaction Between the Mutated GliaMUT Protein and Gliadin-Specific Antibodies, Also in the Certified Serum of a Celiac Patient

a) The effect of amino acid substitutions on the recognition by a specific monoclonal antibody directed to the region of the epitope which activates the immune response in the celiac subject was verified using a commercial enzyme-linked immunosorbent assay (ELISA) currently used for the quantitative analysis of gliadin (wheat), secalin (rye) and hordein (barley) in foods declared gluten-free recognized as AOAC-OMA (2012.01) and AACCI 38.50.0, certified by AOAC-RI (120601) and official method (type 1) of the Codex Alimentarius. Such an assay is based on the use of the R5 monoclonal antibody anti-α-gliadin.

The GliaWT and GliaMUT proteins obtained by alcoholic extraction from the culture pellets (250 ml) of Shuffle cells after 4 hours of induction were analyzed with the RIDASCREEN® GliadinRbiopharm kit, following the indications provided by the manufacturer, before (A) and after (B) purification by IMAC. The extracts (ext) and purified samples (PUR) were assayed at different dilutions (1:12.5 to 1:1600) and GliaWT samples were quantified by referring to the internal standard of purified α-gliadin at known concentration provided by the kit. The absorbance signal was detected by an ELISA reader at a wavelength of 450 nm.

The results obtained on the alcoholic extracts obtained according to the procedure of example 2 and on the purified proteins obtained in example 3, showed that the GliaMUT protein, unlike the GliaWT protein, is not recognized by the monoclonal antibody anti-α-gliadin R5 which binds to the immunoactive portion of gliadin DQ2.

Using a standard gliadin at known concentration, it was possible to determine that under the conditions used, the cells of the SHuffle strain carrying the pET-GliaWT plasmid induced at 30° C. for 4 hours with 0.5 mM IPTG produce 208 μg protein/liter of culture. The protein yield with high degree of purity (>90%) after purification on chromatographic column but under non-optimized conditions is reduced to 50 μg/liter.

The densitometric analysis of a WB in which the quantified GliaWT produced in SHuffle cells, the GliaWT produced in Rosetta and the GliaMUT produced in both SHuffle and Rosetta were loaded allowed concluding that by alcoholic extraction it is possible to obtain:

| | | | |
|---|---|---|---|
| GliaWT SHuffle | (30° C.) | 208 | μg/liter culture |
| GliaWT Rosetta | (37° C.) | 160 | μg/liter culture |
| GliaMUT SHuffle | (30° C.) | 80 | μg/liter culture |
| GliaMUT Rosetta | (37° C.) | 280 | μg/liter culture |

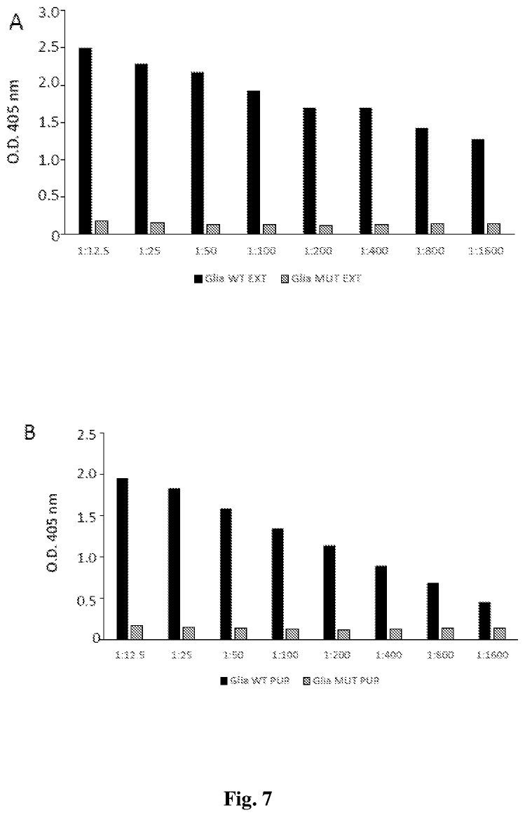

The quantitative analysis data are shown in FIG. 7.
b) The effect of amino acid substitutions on recognition by an anti-gliadin polyclonal rabbit antibody or IgA antibodies present in the serums of celiac subjects was verified by enzyme immunoassay.

The protein extracts obtained from IPTG-induced bacteria which did not express any heterologous protein or which expressed the GliaWT protein or the GliaMUT protein were analyzed by ELISA assays using a commercial polyclonal antibody of rabbit anti-gliadin (G9144 Anti-Gliadin (Wheat) antibody produced in rabbit, SIGMA) or the serum of subjects diagnosed with celiac disease but not yet subjected to a gluten-free diet.

The E. coli protein extracts containing 500 ng Glia WT or 500 ng Glia MUT, or with a control E. coli protein extract containing no heterologous protein, were incubated with:
rabbit anti-gliadin antibody, of which the binding to proteins present in the well was detected by a rabbit immunoglobulin-specific goat secondary antibody and labeled with horseradish peroxidase (31460 Rabbit IgG (H+L) Secondary Antibody HRP, Termofischer)
the serum of a healthy subject (serum C-) or that of a subject diagnosed with celiac disease at different dilutions of (Dil 1, Dil 2 and Dil 3) used as a positive control calibrated by a commercial enzyme immunoassay in colorimetry for the determination of anti α-gliadin specific IgA antibodies in human serum (α-gliatest SIgA Chromo Eurospital).

The results obtained showed that the GliaMUT protein, unlike the GliaWT protein, is recognized with significantly lower efficiency by the rabbit polyclonal anti-gliadin antibody (FIG. 8A) and is not recognized by the IgA present in the serum of the celiac subject (FIG. 8B)

### Example 5—Expression Assays in Nicotiana benthamiana Plants

Suspensions of A. tumefaciens LBA4404 cells transformed with plasmids carrying the sequences encoding the GliaWT and GliaMUT proteins, were used to infiltrate N. benthamiana plants (agroinfiltration) in combination with A. tumefaciens suspensions transformed with a plasmid carrying the sequence encoding the p19 protein (p19: post-transcriptional silencing suppressant derived from Artichoke Mottled Crinckle virus).

Crude protein extracts obtained from agro-infiltrated leaves with the constructs of interest or with pBI-Ω/p19 alone (20 μg total soluble proteins, TSP) were analyzed for a period of 6 days post-infiltration (d.p.i.) to assess the accumulation trend of the recombinant proteins. Both the soluble and insoluble fractions of the leaf extract were analyzed by Western Blot analysis. Briefly, foliar biomass samples were finely ground in liquid N₂ with mortar and pestle, resuspended and homogenized in extraction buffer (200 mg biomass; 1:3 weight/volume; Glycerol Buffer: 100 mM Tris-HCl pH 8.1; 10% glycerol; 400 mM sucrose; 5 mM MgCl₂; 10 mM KCl; 10 mM 2-β-mercaptoethanol) containing a protease inhibitor cocktail (Complete™; Roche, Mannheim, Germany). The samples were incubated on ice for 30 min in slight oscillation and the extracts were clarified by centrifugation at 16,000×g for 20 min. The supernatants were transferred to a new tube and kept on ice until use. The total soluble protein (TSP) content in the supernatants was estimated by Bradford assay (Bio-Rad Inc.). The pellets (insoluble fraction) were resuspended in equal volume of 1× SDS-PAGE sample buffer (10% glycerol, 60 mM Tris-HCl pH 6.8, 0.025% bromophenol blue, 2% SDS, 3% 2-mercaptoethanol). The samples containing 20 μg of TSP and the corresponding insoluble fraction were denatured at 9° C. for 5 minutes and then subjected to polyacrylamide gel electrophoresis (SDS-PAGE 12%), The proteins were electrotransferred from the gel onto PVDF membrane. The membrane was saturated using a 4% (w/v) solution of skim milk in phosphate buffer (PBS). The recombinant proteins were detected by incubating the membrane first with an anti-FLAG tag-HRP antibody diluted 1:1000 in 2% (w/v) skim milk in PBS. The detection was carried out by chemiluminescence with the ECL solution. As a negative control, plants infiltrated only with A. tumefaciens carrying the plasmid encoding the silencing suppressor protein (p19 (−)) were used (the expected band pM ˜35 kDa was highlighted) (FIGS. 9A and 9B). The soluble fraction of the GliaMUT extracts was used to carry out purification by IMAC and the fractions eluted from the chromatographic column analyzed, as previously described, by SDS-PAGE and WB with anti-FLAG tag antibody (FIG. 9C). Overall, these analyses showed that both proteins are produced in soluble form in plant cells and that despite being expressed at lower levels than GliaWT, GliaMUT can be purified.

1. A modified form of the α-gliadin protein comprising amino acid substitutions in at least 80% of positions 13, 18, 23, 25, 30, 36, 37, 42, 49, 54, 55, 64, 67, 75, 79, 81, 89, 93, 115, 123, 130, 139, 144, 151, 164, 181, 191, 197, 200, 205, 211, 218, 224, 230, 232, 240 of the sequence of the predominant mature form, without signal peptide, of α-gliadin of Triticum aestivum (UniProtK-P02863) and having sequence SEQ ID No. 1.

2. The modified form of the α-gliadin protein according to claim 1 comprising the amino acid substitutions at positions 13, 18, 23, 25, 30, 36, 37, 42, 49, 54, 55, 64, 67, 75, 79, 81, 89, 93, 115, 123, 130, 139, 144, 151, 164, 181, 191, 197, 200, 205, 211, 218, 224, 230, 232, 240 of the sequence of the predominant mature form, without signal peptide, of α-gliadin of Triticum aestivum and having sequence SEQ ID No. 1.

3. The modified form of the α-gliadin protein according to claim 2, wherein from 50 to 70% of the substitutions are conservative amino acid substitutions with respect to the sequence of the predominant form of α-gliadin of Triticum aestivum.

4. The modified form of the α-gliadin protein according to claim 3, wherein 64% of the substitutions are conservative amino acid substitutions with respect to the sequence of the predominant form of α-gliadin of Triticum aestivum.

5. A polynucleotide sequence of DNA, deoxyribonucleic acid, encoding a modified form of α-gliadin of Triticum aestivum according to claim 1 having sequence SEQ ID No: 3 or a related polynucleotide sequence thereof due to the degeneration of the genetic code or comprising codons optimized for expression in cell systems or host cells for industrial production.

6. An expression vector comprising the DNA polynucleotide sequence according to claim 5.

7. The expression vector according to claim 6 which is suitable for expression in a prokaryotic microorganism, in a eukaryotic organism, in an animal or plant organism.

8. A cell comprising the polynucleotide sequence encoding the modified α-gliadin protein according to claim 1.

9. The cell according to claim 8, wherein the cell is the cell of a prokaryote, or the cell of a eukaryote, in particular the cell of a grain which endogenously does not contain gluten, a cell of rice (Oryza sativa), oats (Avena sativa), corn (Zea mays), sorghum (Sorghum vulgare), buckwheat (Fagopyrum esculentum), Quinoa (Chenopodium quinoa) or even a cell of a genetically modified wheat species in which the α-gliadin genes have been eliminated.

10. A plant comprising in the genome thereof the polynucleotide sequence encoding the modified α-gliadin protein according to claim 1.

11. A method for producing detoxified or reduced-toxicity foods or processed food products for celiac subjects, comprising combining the modified α-gliadin protein according to claim 1 extracted and/or purified from bacteria, eukaryotic cells and plant cells alone or in addition to flours obtained from plant species which endogenously do not contain gluten, from rice (Oryza sativa), oats (Avena sativa), corn (Zea mays), sorghum (Sorghum vulgare), buckwheat (Fagopyrum esculentum), Quinoa (Chenopodium quinoa) or even from genetically modified wheat species in which the α-gliadin genes have been eliminated.

12. A food or a processed food product comprising modified α-gliadin according to claim 1, detoxified or with reduced toxicity for celiac subjects.

13. The food or food product according to claim 12 obtained through baking.


SEQUENCE LISTING (15 sequences)

SEQ ID NO: 1 - protein, 266 aa, synthetic construct
VRFPVPQLQPQNSSQQQSQEQVGLLQQQQSLGQQQSSPPQQSYPQPQPNPSQLSPLQLQPFPQAQLSYSQPQPFPPQQSYSQPQPQYSKPQQSISQQQQQQQQQQQQQQQQQQIGQQILQQQVIPCMDVTLQQHNIAHPRSQVGQQSTYQGLQELCCQHLWQISEQSQCQAIHNVVHAIIHHQQQKQQQQSSSQVSSQQSLQQYSLGQGSSRPSQQNSQAQGSKQPQQLSQQEEIRNLAKQTLPAMCNVYIPPYCTIAPFGIFGTN

SEQ ID NO: 2 - protein, 266 aa, Triticum aestivum
VRFPVPQLQPQNPSQQQPQEQVPLVQQQQFLGQQQPFPPQQPYPQPQPFPSQLPYLQLQPFPQPQLPYSQPQPFRPQQPYPQPQPQYSQPQQPISQQQQQQQQQQQQQQQQQQILQQILQQQLIPCMDVVLQQHNIAHGRSQVLQQSTYQLLQELCCQHLWQIPEQSQCQAIHNVVHAIILHQQQKQQQQPSSQVSFQQPLQQYPLGQGSFRPSQQNPQAQGSVQPQQLPQFEEIRNLALQTLPAMCNVYIPPYCTIAPFGIFGTN

SEQ ID NO: 3 - DNA, 918 bp, synthetic construct
  source 1..918: mol_type = other DNA
atgaaaaccttcctgattctcgttctcttggcgattgtcgcgactactgcgactactgctgttcggtttcctgttccccaacttcagccgcaaaatagcagccagcagcaatctcaagagcaagtcggtctgctccaacagcagcaatccttgggtcagcagcagtctagtccgcctcaacagtcgtatccgcagccacagccgaatccatcgcagttaagccctcttcaactgcaaccgtttccacaggcacagctgagctattctcaaccgcaaccattcccgcctcaacaaagctattcacagccgcaaccacagtactctaaaccccaacagagcatctctcagcagcaacaacaacaacagcaacaacagcagcagcagcaacagcaacagcagattgggcaacagattctgcagcagcaggtcattccctgtatggatgtcaccctgcaacagcacaacatcgctcatccgcgttcccaggttggccaacaaagcacgtatcagggtctgcaggaactttgttgtcagcatttatggcaaatctcggaacaatcccaatgtcaggcaattcacaacgtggtacatgccattatccatcaccaacaacagaaacagcagcaacagagtagttcacaagtaagctcgcagcagagtctgcaacaatacagccttggtcagggtagttctcgtcccagtcaacagaactcccaggctcagggctccaaacagccgcaacagctgtcgcaacaggaagagattcggaatctggctaagcagacattaccagccatgtgcaatgtctacattccaccctattgcaccattgcacctttcgggatcttcggaacgaacattgaaggacgcgactacaaggacgatgacgataagcatcaccatcatcatcattgataa

SEQ ID NO: 4 - DNA, 918 bp, Triticum aestivum
  misc_feature 1..918: note = gliawt
  source 1..918: mol_type = genomic DNA
atgaaaaccttcttgatcctggttctgttggccattgtagcaacgactgcaacgactgcggtacggtttccggtaccgcagctccagccacagaatccgtcgcaacaacagcctcaggaacaggtaccattagtccagcagcaacagttcttgggacaacagcagccatttccgccccaacaaccctatccgcagccgcagccgtttccctcccaacttccgtatctccagttacaaccgtttcctcagcctcagttaccgtactctcagccgcaacccttccgtccgcaacaaccttatccccaaccacaaccgcagtattctcagccacaacaaccgatctcacagcagcagcagcaacaacagcagcaacaacagcaacagcaacaacaacaacagattctgcagcaaatcctgcagcaacagctgattccctgtatggatgtggtgttgcaacagcacaacattgctcatggtcgctcacaggttctgcagcagtcaacgtaccagttactgcaggaactgtgttgccaacatctgtggcagatcccggaacagtctcagtgtcaggcgatccataacgtcgttcatgcgatcatccttcatcagcaacagaagcaacagcagcagccttcaagccaagtgtcgtttcagcaaccactgcagcaatacccattaggccagggttcctttcgcccaagccaacagaatcctcaggctcaagggagtgttcaaccgcagcaactgccacagttcgaggagattcgcaatctggcgctgcagaccttacccgcaatgtgtaacgtgtacattccgccgtattgcaccattgccccgtttggcattttcggcacaaacatcgaaggtcgcgattacaaagacgatgatgacaaacaccatcatcaccatcattgataa

SEQ ID NO: 5 - protein, 9 aa, synthetic construct
  REGION 1..9: note = Prior Art Sequence
PFPQPZLPY

SEQ ID NO: 6 - protein, 9 aa, synthetic construct
  REGION 1..9: note = Prior Art Sequence
PYPQPZLPY

SEQ ID NO: 7 - protein, 10 aa, synthetic construct
  REGION 1..10: note = Prior Art Sequence
PPQPZLPYPQ

SEQ ID NO: 8 - protein, 10 aa, synthetic construct
  REGION 1..10: note = Prior Art Sequence
FPQPZLPYPQ

SEQ ID NO: 9 - protein, 9 aa, synthetic construct
  REGION 1..9: note = Prior Art Sequence
FRPQQPYPQ

SEQ ID NO: 10 - protein, 9 aa, synthetic construct
  REGION 1..9: note = Prior Art Sequence
QGSFQPSQQ

SEQ ID NO: 11 - protein, 7 aa, synthetic construct
  REGION 1..7: note = Prior Art Sequence
QLPYPQP

SEQ ID NO: 12 - protein, 7 aa, synthetic construct
  REGION 1..7: note = Prior Art Sequence
QLPYSQP

SEQ ID NO: 13 - protein, 7 aa, synthetic construct
  REGION 1..7: note = Prior Art Sequence
PLPYPQP

SEQ ID NO: 14 - protein, 304 aa, synthetic construct
  REGION 1..304: note = Recombinant
MKTFLILVLLAIVATTATTAVRFPVPQLQPQNSSQQQSQEQVGLLQQQQSLGQQQSSPPQQSYPQPQPNPSQLSPLQLQPFPQAQLSYSQPQPFPPQQSYSQPQPQYSKPQQSISQQQQQQQQQQQQQQQQQQIGQQILQQQVIPCMDVTLQQHNIAHPRSQVGQQSTYQGLQELCCQHLWQISEQSQCQAIHNVVHAIIHHQQQKQQQQSSSQVSSQQSLQQYSLGQGSSRPSQQNSQAQGSKQPQQLSQQEEIRNLAKQTLPAMCNVYIPPYCTIAPFGIFGTNIEGRDYKDDDDKHHHHHH

SEQ ID NO: 15 - protein, 304 aa, synthetic construct
  REGION 1..304: note = Recombinant
MKTFLILVLLAIVATTATTAVRFPVPQLQPQNPSQQQPQEQVPLVQQQQFLGQQQPFPPQQPYPQPQPFPSQLPYLQLQPFPQPQLPYSQPQPFRPQQPYPQPQPQYSQPQQPISQQQQQQQQQQQQQQQQQQILQQILQQQLIPCMDVVLQQHNIAHGRSQVLQQSTYQLLQELCCQHLWQIPEQSQCQAIHNVVHAIILHQQQKQQQQPSSQVSFQQPLQQYPLGQGSFRPSQQNPQAQGSVQPQQLPQFEEIRNLALQTLPAMCNVYIPPYCTIAPFGIFGTNIEGRDYKDDDDKHHHHHH